(19) United States
(12) Patent Application Publication
(10) Pub. No.: US 2025/0020826 A1
(43) Pub. Date: Jan. 16, 2025

(54) EARLY WARNING METHOD FOR SHALLOW SOIL LANDSLIDE BASED ON RAINFALL AND SOIL MOISTURE CONTENT

(71) Applicants: Chengdu University of Technology, Chengdu (CN); Guizhou Geological Environment Monitoring Institute (Guizhou Institute of Environmental Geology), Guiyang (CN)

(72) Inventors: Bin Yu, Chengdu (CN); Yangchun Li, Guiyang (CN); Wenhong Chen, Chengdu (CN)

(21) Appl. No.: 18/899,626

(22) Filed: Sep. 27, 2024

(30) Foreign Application Priority Data
Oct. 31, 2023 (CN) 202311429947.X

(51) Int. Cl. G01V 9/00; G01N 33/24; G08B 21/10

(52) U.S. Cl. CPC G01V 9/00; G01N 33/246; G08B 21/10

(57) ABSTRACT

The present disclosure discloses an early warning method for a shallow soil landslide based on rainfall and soil moisture content, relating to the field of soil landslides prevention and control. The early warning method comprises the following steps: a. determining whether a location is a humid region; b. calculating soil moisture content of the humid region in a rainy season; c. monitoring rainfall amounts in real time by sensors; d. calculating the soil moisture content S by step b; e. calculating rainfall duration on an hourly basis, calculating a total hourly rainfall amount by superposition from an initial 1-hour rainfall amount, and dividing the total hourly rainfall amount by the rainfall duration to obtain hourly rainfall intensity; f. calculating a critical value Cr of a shallow soil landslide; and g. determining a landslide grade and giving an early warning signal.

### TECHNICAL FIELD

The present disclosure relates to the technical field of soil landslides prevention and control, in particular to an early warning method for a shallow soil landslide based on rainfall and soil moisture content.

### BACKGROUND

In mountainous regions, landslides often occur in rainy seasons, and soil landslides are a natural phenomenon that occurs in mountainous regions or road slopes. After a soil landslide occurs, sliding soil moves down the slope or onto the roadside, burying and impacting building facilities, roads, and other infrastructure, resulting in significant damage.

Most landslides in China are caused by rainfall. Therefore, many early warning and prediction systems for landslides are implemented by rainfall conditions. At present, the most widely used early warning models worldwide are based on rainfall intensity and rainfall duration. However, the two factors inevitably involve rainfall onset time and whether previous rainfall affects subsequent landslides. That is, defining a rainfall threshold or a segmentation method is required to distinguish between rainfall that has no impact on landslides and rainfall that does. Due to the wide range of rainfall duration indexes, spanning from 0.2 to 1.6, it is challenging to establish a unified early warning model, and critical values also vary greatly, mainly related to a starting point and an ending point of rainfall.

At present, forecasting landslides at home and abroad is mainly based on the impact of rainfall on slopes, and many models have been proposed. However, these models lack a clear description of the starting point and ending point of rainfall, and therefore, have significant limitations in their application.

CN104299367A published on Jan. 21, 2015 discloses a multi-level comprehensive monitoring and early warning method for landslide disasters, including the following steps: (1) calculating a critical threshold of landslide monitoring and early warning through historical recording and monitoring data and landslide deformation and failure model experiments; and determining, based on a critical index of each indicator, whether there is a possibility of landslide occurrence in a study region; (2) in case of a monitoring value greater than the critical threshold, determining a possible location and possibility scale of landslide occurrence based on each landslide occurrence index, and establishing early warning and forecasting levels; (3) determining four early warning levels and corresponding early warning boundary regions; and (4) publishing early warning results.

The multi-level comprehensive monitoring and early warning method for landslide disasters disclosed in the patent document requires extensive historical recording and monitoring data of landslide occurrence, the critical threshold of landslide monitoring and early warning is calculated through landslide deformation and failure model experiments, and then whether there is a possibility of landslide occurrence in the study region is determined based on the critical index of each indicator. Involving so many landslide factors, the entire process is complex, with a delayed response, low early warning efficiency, and poor disaster prevention applicability.

CN101598721A published on Dec. 9, 2009 discloses a method for predicting and forecasting stability of a soil slope under rainfall conditions, including: firstly, establishing a comprehensive table of control factors for soil slope stability through 11 parameters; then establishing a flood resistance capability rating table for each control factor by mathematical regression analysis on the soil slope stability control factors; obtaining an effective rainfall threshold during landslides by investigation and statistical analysis on the relationship between regional landslides and rainfall; and finally establishing a stability warning level map for soil slopes under rainfall conditions based on the flood resistance capability rating table combined with various rainfall patterns and characteristics, to predict and forecast the stability state of a specific slope based on the stability warning level map.

The method for predicting and forecasting the stability of a soil slope under rainfall conditions disclosed in the patent document does not consider the lagged impact of rainfall or extreme rainfall on the stability of a slope under different duration conditions, and impedes the timely response to the early warning of landslides due to the lagged prediction on landslide instability, resulting in poor disaster prevention applicability.

CN105701975A published on Jun. 22, 2016 discloses an early warning method for a shallow soil landslide in a red-bed region, including the following steps: designating a landslide mass and a non-landslide mass in a red-bed region as investigation objects, and measuring their slope gradient, landslide mass area, upper slope gradient, upper area, lateral left slope gradient, lateral right slope gradient, lateral left area, and lateral right area, and terraced area of the landslide mass; calculating a slope gradient factor, an upper side factor, two side factors, a terrain factor, and a rainfall factor, calculating a critical value for inducing shallow soil landslides in the red-bed region, performing conditional discrimination to determine a landslide grade, and giving an early warning signal.

The early warning method for a shallow soil landslide disclosed in the patent document does not specifically propose a starting point and an ending point of rainfall, preventing the accurate determination of rainfall onset. Although the method may function adequately during sudden rainfall following extended dry periods, it faces challenges in rainy seasons that induce landslides due to intermittent rains. In such cases, if the rainfall accumulations before heavy rainfall are not included, the impact of previous rainfall may be underestimated, resulting in underreporting of potential landslides. Conversely, if all of the rainfall accumulations before heavy rainfall are included, the impact of previous rainfall may be overestimated, resulting in false forecasting.

### SUMMARY

In order to overcome the above shortcomings of the prior art, the present disclosure provides an early warning method for a shallow soil landslide based on rainfall and soil moisture content. The present disclosure fully considers a combined effect of rainfall and previous rainfall, determines, by soil moisture content, a starting point of rainfall that affects landslides, and then uses a rainfall model to form a forecasting model for rainfall-induced shallow soil landslides, so that the obtained critical value of a shallow soil landslide is more in line with a mechanism of inducing shallow landslides. This early warning method does not require extensive historical observation data of landslide occurrence. Not only does it provide a highly accurate landslide early warning, but it also greatly shortens the early warning response time and improves the applicability of disaster prevention.

The present disclosure is implemented by the following technical solution:

An early warning method for a shallow soil landslide based on rainfall and soil moisture content includes the following steps:
a. consulting meteorological data for a location of a landslide mass to obtain a local annual average rainfall amount, local annual average sunshine duration, and a local average temperature in a rainy season from May to September, and determining whether the location is a humid region according to Equation 1;

$$Rm > 1.38 Ss - 1068 \qquad Equation 1$$

where Rm is the annual average rainfall amount in mm; and Ss is the annual average sunshine duration in h;
b. calculating soil moisture content of the humid region in the rainy season;

$$S = 0.0168 ln ( M ) + 0.3452 \qquad Equation 2$$

where S is the soil moisture content; and M is a soil moisture index, calculated by Equation 3;

$$M = R / pe \qquad Equation 3$$

where R is a local 24-hour rainfall amount in mm/d; and Pe is maximum latent heat evaporation in mm/d, calculated by Equation 4;

$$pe = \{ \begin{matrix}0 & {T}_{a} < 0 ° C . \\ [ 0.533 {( \frac{10 {T}_{a}}{I} )}^{a} ] \frac{h}{12} & 0 ° C . \leq {T}_{a} \leq 26.5 ° C . \\ - 13.86 + 1.075 {T}_{a} - 0.0144 {T}_{a}^{2} & {T}_{a} > 26.5 ° C .\end{matrix} i . \qquad Equation 4$$

where Tₐ is a monthly average temperature in ° C.; I is an annual total heating index, calculated by Equation 5; H is daylight length in h; and a is a coefficient, calculated by Equation 6;

$$\begin{matrix}I = {\sum }_{1}^{12} i & Equation 5 \\ a = 0.492 + 1.792 \times {10}^{- 2} I - 7.71 \times {10}^{- 5} {I}^{2} + 6.75 \times {10}^{- 7} {I}^{3} & Equation 6\end{matrix}$$

where i is an annual heating coefficient, calculated by Equation 7;

$$i = {( \frac{{T}_{a}}{5} )}^{1.514} ; \qquad Equation 7$$

c. monitoring rainfall amounts in real time by a plurality of sensors at a monitored landslide site, and obtaining a first 24-hour rainfall amount on an hourly basis;
d. calculating the soil moisture content S by step b, where if S<0.37, rainfall has no impact on the landslide and is disregarded, rainfall duration D=0, and the rainfall amount is 0; if S≥0.37, an onset of the rainfall is confirmed, the rainfall duration D=1 h, and a previous 1-hour rainfall amount is included in a total rainfall amount as an initial 1-hour rainfall amount; and if the soil moisture content S<0.37 again, the previous rainfall amount no longer affects the landslide, the rainfall duration D=0, and the rainfall amount is 0;
e. when D=1 h in step d, calculating the rainfall duration on an hourly basis, and calculating a total hourly rainfall amount by superposition from the initial 1-hour rainfall amount, and dividing the total hourly rainfall amount by the rainfall duration to obtain hourly rainfall intensity;
f. calculating a critical value Cr of a shallow soil landslide by Equation 8 based on the rainfall duration and the rainfall intensity;

$$C r = {I}_{i} {D}^{0.921} \qquad Equation 8$$

where Iᵢ is the rainfall intensity in mm/h; and D is the rainfall duration in h; and

g. performing conditional discrimination based on the critical value Cr of the shallow soil landslide obtained in step f to determine a landslide grade, and giving an early warning signal.

In step g, the landslide grade is sequentially a green early warning grade, a yellow early warning grade, an orange early warning grade, and a red early warning grade.

In step g, the early warning signal is determined by the conditional discrimination: when the critical value Cr<32.6, a possibility of a landslide is low, and a green safety signal is given; when 32.6≤Cr<61.7, the possibility is moderate, and a yellow early warning signal is given; when 61.7≤Cr<90.8, the possibility is high, and an orange early warning signal is given; and when the critical value Cr≥90.8, the possibility is very high, and a red early warning signal is given.

The basic principle of the present disclosure is as follows:

The main factor that induces shallow soil landslides is rainfall, with key parameters being rainfall duration and rainfall intensity. To determine the rainfall duration and the rainfall intensity for the early warning of landslides, it is crucial to first determine the onset time of rainfall, i.e., the time when rainfall starts to affect landslides. In the present disclosure, a more direct factor—the soil moisture content—is used as to identify the time of rainfall that affects landslides, thereby overcoming the indirect effect of rainfall on landslides and soil and more directly expressing the impact of rainfall on soil. Furthermore, the value of the soil moisture content reflects the impact of rainfall, that is, when the soil moisture content reaches a certain level, rainfall starts to take effect, and subsequent heavy rainfall will affect the occurrence of landslides, thereby greatly improving the accuracy of early warning of rainfall-induced landslides. Meanwhile, the interception of rainwater by vegetation on the slope and the interception of rainwater by surface soil are excluded, so that the rainfall duration and the rainfall intensity are parameters that can directly affect landslides, thereby improving the accuracy of early warning.

The beneficial effects of the present disclosure are mainly manifested in the following aspects:

1. The present disclosure fully considers a combined effect of rainfall and previous rainfall, determines, by soil moisture content, a starting point of rainfall that affects landslides, and then uses a rainfall model to form a forecasting model for rainfall-induced shallow soil landslides, so that the obtained critical value of a shallow soil landslide is more in line with a mechanism of inducing shallow landslides. This early warning method does not require extensive historical observation data of landslide occurrence. Not only does it provide a highly accurate landslide early warning, but it also greatly shortens the early warning response time and improves the applicability of disaster prevention.

2. When applied to the early warning of shallow soil landslides, compared with the prior art, the present disclosure obtains a critical value Cr of a shallow soil landslide more in line with the actual situation and practicality of inducing shallow landslides, and the accuracy of the early warning of landslides is higher, in that the calculation on the critical value Cr simultaneously considers the effects of the rainfall starting point, the rainfall duration, and the rainfall intensity. In addition, for a given unstable slope, the calculation on the critical value Cr of a shallow soil landslide using the method of the present disclosure only requires rainfall process data of the observed region, which can make the fastest response to the early warning of the shallow soil landslide and greatly improve the disaster prevention capability.

3. The present disclosure fully considers the rainfall starting point determined by the soil moisture content, then obtains the rainfall duration and the rainfall intensity under certain rainfall conditions that affect the soil mass, and further calculates the critical value Cr of the shallow soil landslide and performs conditional discrimination, so that the possibility scale of the shallow soil landslide in a region can be quickly determined. Compared with the prior art that only considers rainfall conditions for the early warning of landslides, the present disclosure not only has higher accuracy, but also can make the fastest response, with high early warning sensitivity and robust disaster prevention applicability.

4. The rainfall starting point is determined by calculating the hourly soil moisture content, and the rainfall amount is obtained by an analysis on the rainfall process and the occurrence of landslides. Therefore, the present disclosure is not constrained by geographical limitations and has a wider application range.

5. Rainfall is the primary trigger for landslides, and the stimulation of landslides is also caused by rainfall. Rainfall reduces the infiltration rate of soil, increases the water content in slope soil, raises the groundwater level, and increases the pore water pressure, thereby reducing the stability of the slope and inducing landslides under the action of heavy rainfall. By calculating the rainfall starting point based on the soil moisture content, rainfall that stimulates landslides and rainfall that is unrelated to landslides in humid regions can be effectively distinguished in the present disclosure, facilitating the accurate prediction of the occurrence of landslides.

6. Compared with the prior art, the rainfall starting point in the present disclosure is not calculated based on the occurrence of a large number of collapses and landslides in strong earthquake regions. Therefore, the present disclosure is applicable for regions with no or small earthquake impact, as well as during extended periods after earthquakes, and has wide applicability for calculating the rainfall that induces landslides.

7. The present disclosure overcomes the indirect effect of rainfall on landslides and soil and more directly expresses the impact of rainfall on soil. Furthermore, the value of the soil moisture content reflects the impact of rainfall, that is, when the soil moisture content reaches a certain level, rainfall starts to take effect, and subsequent heavy rainfall will affect the occurrence of landslides, thereby greatly improving the accuracy of early warning of rainfall-induced landslides.

8. The soil moisture content, the rainfall starting point, the rainfall duration, and the rainfall intensity in the present disclosure are calculated by a little measured data, and extensive historical observation data on the occurrence of landslides is not required, thereby greatly shortening the early warning response time.

9. In the present disclosure, conditional discrimination is performed on the calculated critical value Cr of the shallow soil landslide to determine a landslide grade, and the corresponding early warning signal is given. When the critical value Cr<32.6, the possibility of a landslide is low, and a green safety signal is given; when 32.6≤Cr<61.7, the possibility is moderate, and a yellow early warning signal is given; when 61.7≤Cr<90.8, the possibility is high, and an orange early warning signal is given; and when the critical value Cr≥90.8, the possibility is very high, and a red early warning signal is given. In this way, the early warning effect is intuitive and clear, with high precision, greatly improving the applicability of disaster prevention.

### DETAILED DESCRIPTION OF THE EMBODIMENTS

### Example 1

An early warning method for a shallow soil landslide based on rainfall and soil moisture content includes the following steps:
a. consulting meteorological data for a location of a landslide mass to obtain a local annual average rainfall amount, local annual average sunshine duration, and a local average temperature in a rainy season from May to September, and determining whether the location is a humid region according to Equation 1;

$$Rm > 1.38 Ss - 1068 \qquad Equation 1$$

where Rm is the annual average rainfall amount in mm; and Ss is the annual average sunshine duration in h;
b. calculating soil moisture content of the humid region in the rainy season;

$$S = 0.0168 ln ( M ) + 0.3452 \qquad Equation 2$$

where S is the soil moisture content; and M is a soil moisture index, calculated by Equation 3;

$$M = R / pe \qquad Equation 3$$

where R is a local 24-hour rainfall amount in mm/d; and Pe is maximum latent heat evaporation in mm/d, calculated by Equation 4;

$$i .$$

$$pe = \{ \begin{matrix}0 & {T}_{a} < 0 ° C . \\ [ 0.533 {( \frac{10 {T}_{a}}{I} )}^{a} ] \frac{h}{12} & 0 ° C . \leq {T}_{a} \leq 26.5 ° C . \\ - 13.86 + 1.075 {T}_{a} - 0.0144 {T}_{a}^{2} & {T}_{a} > 26.5 ° C .\end{matrix} \qquad Equation 4$$

where Tₐ is a monthly average temperature in ° C.; I is an annual total heating index, calculated by Equation 5; H is daylight length in h; and a is a coefficient, calculated by Equation 6;

$$I = {\sum }_{1}^{12} i \qquad Equation 5$$

$$a = 0. 492 + 1.792 \times 1 {0}^{- 2} I - 7.71 \times 1 {0}^{- 5} {I}^{2} + 6.75 \times 1 {0}^{- 7} {I}^{3} \qquad Equation 6$$

where i is an annual heating coefficient, calculated by Equation 7;

$$i = {( \frac{{T}_{a}}{5} )}^{1.514} ; \qquad Equation 7$$

c. monitoring rainfall amounts in real time by a plurality of sensors at a monitored landslide site, and obtaining a first 24-hour rainfall amount on an hourly basis;
d. calculating the soil moisture content S by step b, where if S<0.37, rainfall has no impact on the landslide and is disregarded, rainfall duration D=0, and the rainfall amount is 0; if S≥0.37, an onset of the rainfall is confirmed, the rainfall duration D=1 h, and a previous 1-hour rainfall amount is included in a total rainfall amount as an initial 1-hour rainfall amount; and if the soil moisture content S<0.37 again, the previous rainfall amount no longer affects the landslide, the rainfall duration D=0, and the rainfall amount is 0;
e. when D=1 h in step d, calculating the rainfall duration on an hourly basis, and calculating a total hourly rainfall amount by superposition from the initial 1-hour rainfall amount, and dividing the total hourly rainfall amount by the rainfall duration to obtain hourly rainfall intensity;
f. calculating a critical value Cr of a shallow soil landslide by Equation 8 based on the rainfall duration and the rainfall intensity;

$$C r = {I}_{i} {D}^{0.921} \qquad Equation 8$$

where Iᵢ is the rainfall intensity in mm/h; and D is the rainfall duration in h; and
g. performing conditional discrimination based on the critical value Cr of the shallow soil landslide obtained in step f to determine a landslide grade, and giving an early warning signal.

This example is the most basic embodiment, which fully considers a combined effect of rainfall and previous rainfall, determines, by soil moisture content, a starting point of rainfall that affects landslides, and then uses a rainfall model to form a forecasting model for rainfall-induced shallow soil landslides, so that the obtained critical value of a shallow soil landslide is more in line with a mechanism of inducing shallow landslides. This early warning method does not require extensive historical observation data of landslide occurrence. Not only does it provide a highly accurate landslide early warning, but it also greatly shortens the early warning response time and improves the applicability of disaster prevention.

When applied to the early warning of shallow soil landslides, compared with the prior art, the present disclosure obtains a critical value Cr of a shallow soil landslide more in line with the actual situation and practicality of inducing shallow landslides, and the accuracy of the early warning of landslides is higher, in that the calculation on the critical value Cr simultaneously considers the effects of the rainfall starting point, the rainfall duration, and the rainfall intensity. In addition, for a given unstable slope, the calculation on the critical value Cr of a shallow soil landslide using the method of the present disclosure only requires rainfall process data of the observed region, which can make the fastest response to the early warning of the shallow soil landslide and greatly improve the disaster prevention capability.

### Example 2

An early warning method for a shallow soil landslide based on rainfall and soil moisture content includes the following steps:
a. consulting meteorological data for a location of a landslide mass to obtain a local annual average rainfall amount, local annual average sunshine duration, and a local average temperature in a rainy season from May to September, and determining whether the location is a humid region according to Equation 1;

$$Rm > 1.38 Ss - 1068 \qquad Equation 1$$

where Rm is the annual average rainfall amount in mm; and Ss is the annual average sunshine duration in h;
b. calculating soil moisture content of the humid region in the rainy season;

$$S = 0.0168 ln ( M ) + 0.3452 \qquad Equation 2$$

where S is the soil moisture content; and M is a soil moisture index, calculated by Equation 3;

$$M = R / pe \qquad Equation 3$$

where R is a local 24-hour rainfall amount in mm/d; and Pe is maximum latent heat evaporation in mm/d, calculated by Equation 4;

$$i .$$

$$pe = \{ \begin{matrix}0 & {T}_{a} < 0 ° C . \\ [ 0.533 {( \frac{10 {T}_{a}}{I} )}^{a} ] \frac{h}{12} & 0 ° C . \leq {T}_{a} \leq 26.5 ° C . \\ - 13.86 + 1.075 {T}_{a} - 0.0144 {T}_{a}^{2} & {T}_{a} > 26.5 ° C .\end{matrix} \qquad Equation 4$$

where Tₐ is a monthly average temperature in ° C.; I is an annual total heating index, calculated by Equation 5; H is daylight length in h; and a is a coefficient, calculated by Equation 6;

$$I = {\sum }_{1}^{12} i \qquad Equation 5$$

$$a = 0. 492 + 1.792 \times 1 {0}^{- 2} I - 7.71 \times 1 {0}^{- 5} {I}^{2} + 6.75 \times 1 {0}^{- 7} {I}^{3} \qquad Equation 6$$

where i is an annual heating coefficient, calculated by Equation 7;

$$i = {( \frac{{T}_{a}}{5} )}^{1.514} \qquad Equation 7$$

c. monitoring rainfall amounts in real time by a plurality of sensors at a monitored landslide site, and obtaining a first 24-hour rainfall amount on an hourly basis;
d. calculating the soil moisture content S by step b, where if S<0.37, rainfall has no impact on the landslide and is disregarded, rainfall duration D=0, and the rainfall amount is 0; if S≥0.37, an onset of the rainfall is confirmed, the rainfall duration D=1 h, and a previous 1-hour rainfall amount is included in a total rainfall amount as an initial 1-hour rainfall amount; and if the soil moisture content S<0.37 again, the previous rainfall amount no longer affects the landslide, the rainfall duration D=0, and the rainfall amount is 0;
e. when D=1 h in step d, calculating the rainfall duration on an hourly basis, and calculating a total hourly rainfall amount by superposition from the initial 1-hour rainfall amount, and dividing the total hourly rainfall amount by the rainfall duration to obtain hourly rainfall intensity;
f. calculating a critical value Cr of a shallow soil landslide by Equation 8 based on the rainfall duration and the rainfall intensity;

$$C r = {I}_{i} {D}^{0.921} \qquad Equation 8$$

where Iᵢ is the rainfall intensity in mm/h; and D is the rainfall duration in h; and
g. performing conditional discrimination based on the critical value Cr of the shallow soil landslide obtained in step f to determine a landslide grade, and giving an early warning signal.

In step g, the landslide grade is sequentially a green early warning grade, a yellow early warning grade, an orange early warning grade, or a red early warning grade.

This example is a preferred embodiment, which fully considers the rainfall starting point determined by the soil moisture content, then obtains the rainfall duration and the rainfall intensity under certain rainfall conditions that affect the soil mass, and further calculates the critical value Cr of the shallow soil landslide and performs conditional discrimination, so that the possibility scale of the shallow soil landslide in a region can be quickly determined. Compared with the prior art that only considers rainfall conditions for the early warning of landslides, the present disclosure not only has higher accuracy, but also can make the fastest response, with high early warning sensitivity and robust disaster prevention applicability.

The rainfall starting point is determined by calculating the hourly soil moisture content, and the rainfall amount is obtained by an analysis on the rainfall process and the occurrence of landslides. Therefore, the present disclosure is not constrained by geographical limitations and has a wider application range.

Rainfall is the primary trigger for landslides, and the stimulation of landslides is also caused by rainfall. Rainfall reduces the infiltration rate of soil, increases the water content in slope soil, raises the groundwater level, and increases the pore water pressure, thereby reducing the stability of the slope and inducing landslides under the action of heavy rainfall. By calculating the rainfall starting point based on the soil moisture content, rainfall that stimulates landslides and rainfall that is unrelated to landslides in humid regions can be effectively distinguished in the present disclosure, facilitating the accurate prediction of the occurrence of landslides.

### Example 3

An early warning method for a shallow soil landslide based on rainfall and soil moisture content includes the following steps:
a. consulting meteorological data for a location of a landslide mass to obtain a local annual average rainfall amount, local annual average sunshine duration, and a local average temperature in a rainy season from May to September, and determining whether the location is a humid region according to Equation 1;

$$Rm > 1.38 Ss - 1068 \qquad Equation 1$$

where Rm is the annual average rainfall amount in mm; and Ss is the annual average sunshine duration in h;
b. calculating soil moisture content of the humid region in the rainy season;

$$S = 0.0168 ln ( M ) + 0.3452 \qquad Equation 2$$

where S is the soil moisture content; and M is a soil moisture index, calculated by Equation 3;

$$M = R / pe \qquad Equation 3$$

where R is a local 24-hour rainfall amount in mm/d; and Pe is maximum latent heat evaporation in mm/d, calculated by Equation 4;

$$pe = \{ \begin{matrix}0 & {T}_{a} < 0 ° C . \\ [ 0.533 {( \frac{10 {T}_{a}}{I} )}^{a} ] \frac{h}{12} & 0 ° C . \\ - 13.86 + 1.075 {T}_{a} - 0.0144 {T}_{a}^{2} & {T}_{a} > 26.5 ° C .\end{matrix} i . \qquad Equation 4$$

where Tₐ is a monthly average temperature in ° C.; I is an annual total heating index, calculated by Equation 5; H is daylight length in h; and a is a coefficient, calculated by Equation 6;

$$I = {\sum }_{1}^{12} i \qquad Equation 5$$

$$a = 0. 492 + 1.792 \times 1 {0}^{- 2} I - 7.71 \times 1 {0}^{- 5} {I}^{2} + 6.75 \times 1 {0}^{- 7} {I}^{3} \qquad Equation 6$$

where i is an annual heating coefficient, calculated by Equation 7;

$$i = {( \frac{{T}_{a}}{5} )}^{1.514} \qquad Equation 7$$

c. monitoring rainfall amounts in real time by a plurality of sensors at a monitored landslide site, and obtaining a first 24-hour rainfall amount on an hourly basis;
d. calculating the soil moisture content S by step b, where if S<0.37, rainfall has no impact on the landslide and is disregarded, rainfall duration D=0, and the rainfall amount is 0; if S≥0.37, an onset of the rainfall is confirmed, the rainfall duration D=1 h, and a previous 1-hour rainfall amount is included in a total rainfall amount as an initial 1-hour rainfall amount; and if the soil moisture content S<0.37 again, the previous rainfall amount no longer affects the landslide, the rainfall duration D=0, and the rainfall amount is 0;
e. when D=1 h in step d, calculating the rainfall duration on an hourly basis, and calculating a total hourly rainfall amount by superposition from the initial 1-hour rainfall amount, and dividing the total hourly rainfall amount by the rainfall duration to obtain hourly rainfall intensity;
f. calculating a critical value Cr of a shallow soil landslide by Equation 8 based on the rainfall duration and the rainfall intensity;

$$C r = {I}_{i} {D}^{0.921} \qquad Equation 8$$

where Iᵢ is the rainfall intensity in mm/h; and D is the rainfall duration in h; and
g. performing conditional discrimination based on the critical value Cr of the shallow soil landslide obtained in step f to determine a landslide grade, and giving an early warning signal.

In step g, the landslide grade is sequentially a green early warning grade, a yellow early warning grade, an orange early warning grade, or a red early warning grade.

In step g, the early warning signal is determined by the conditional discrimination: when the critical value Cr<32.6, a possibility of a landslide is low, and a green safety signal is given; when 32.6≤Cr<61.7, the possibility is moderate, and a yellow early warning signal is given; when 61.7≤Cr<90.8, the possibility is high, and an orange early warning signal is given; and when the critical value Cr≥90.8, the possibility is very high, and a red early warning signal is given.

This example is the best embodiment. Compared with the prior art, the rainfall starting point in the present disclosure is not calculated based on the occurrence of a large number of collapses and landslides in strong earthquake regions. Therefore, the present disclosure is applicable for regions with no or small earthquake impact, as well as during extended periods after earthquakes, and has wide applicability for calculating the rainfall that induces landslides.

The present disclosure overcomes the indirect effect of rainfall on landslides and soil and more directly expresses the impact of rainfall on soil. Furthermore, the value of the soil moisture content reflects the impact of rainfall, that is, when the soil moisture content reaches a certain level, rainfall starts to take effect, and subsequent heavy rainfall will affect the occurrence of landslides, thereby greatly improving the accuracy of early warning of rainfall-induced landslides.

The soil moisture content, the rainfall starting point, the rainfall duration, and the rainfall intensity in the present disclosure are calculated by a little measured data, and extensive historical observation data on the occurrence of landslides is not required, thereby greatly shortening the early warning response time.

In the present disclosure, conditional discrimination is performed on the calculated critical value Cr of the shallow soil landslide to determine a landslide grade, and the corresponding early warning signal is given. When the critical value Cr<32.6, the possibility of a landslide is low, and a green safety signal is given; when 32.6≤Cr<61.7, the possibility is moderate, and a yellow early warning signal is given; when 61.7≤Cr<90.8, the possibility is high, and an orange early warning signal is given; and when the critical value Cr≥90.8, the possibility is very high, and a red early warning signal is given. In this way, the early warning effect is intuitive and clear, with high precision, greatly improving the applicability of disaster prevention.

The present disclosure is described below with reference to a specific example.

85 shallow soil landslide events occurred with exact time in Guizhou Province from 2016 to 2020. 80 rainfall observation stations were near the landslides, and each landslide event corresponded to one rainfall station. 5 landslides were located close to the other landslides, sharing data from the same rainfall station and occurring at similar time. Guizhou has an average annual rainfall amount ranging from 688 to 1,480 mm, and average annual sunshine duration between 1,000 and 1,500 h, classifying it as a humid region. Meteorological data were consulted for locations of landslide masses, to obtain basic data about an average temperature of local landslides on that month and sunshine duration before and after the landslides occurred; hourly soil moisture content was calculated based on the rainfall data collected for the 7 days before and 1 day after the landslides occurred, rainfall onset time and time when D=1 h were determined based on a critical value of soil moisture content of 0.37, a corresponding rainfall amount and corresponding rainfall intensity were obtained. This process was continued until the time of landslide occurrence, during which the soil moisture content value remained at 0.37 or above, and a critical value of a shallow soil landslide and a possibility of landslide occurrence were calculated. The results were compared with those of actual landslides, with specific values shown in Table 1, which is a table about early warning of shallow soil landslides in Guizhou Province from 2016 to 2020.

TABLE 1

| | | | | | | | | | | Possibility | |
|---|---|---|---|---|---|---|---|---|---|---|---|
| | | | | Possibility | | | | | | of | |
| No. | D | Iᵢ | Cr | of landslides | Occurrence | No. | D | Iᵢ | Cr | landslides | Occurrence |
| 1 | 11 | 6.573 | 59.8 | Moderate | Yes | 41 | 42 | 3.46 | 108 | Very high | Yes |
| 2 | 12 | 21.55 | 213 | Very high | Yes | 42 | 29 | 7.91 | 176 | Very high | Yes |
| 3 | 8 | 20.53 | 139 | Very high | Yes | 43 | 29 | 9.14 | 203 | Very High | Yes |
| 4 | 13 | 10.4 | 110 | Very high | Yes | 44 | 8 | 10.01 | 67.9 | High | Yes |
| 5 | 15 | 12.69 | 154 | Very high | Yes | 45 | 5 | 17.02 | 74.9 | High | Yes |
| 6 | 12 | 23.93 | 236 | Very high | Yes | 46 | 6 | 16.58 | 86.3 | High | Yes |
| 7 | 12 | 18.38 | 181 | Very high | Yes | 47 | 15 | 4.81 | 58.3 | Moderate | Yes |
| 8 | 15 | 16.64 | 202 | Very high | Yes | 48 | 6 | 13.5 | 70.3 | High | Yes |
| 9 | 9 | 17.28 | 131 | Very high | Yes | 49 | 28 | 4.35 | 93.6 | Very high | Yes |
| 10 | 12 | 20.88 | 206 | Very high | Yes | 50 | 29 | 3.5 | 77.8 | High | Yes |
| 11 | 6 | 17.92 | 93.3 | Very high | Yes | 51 | 51 | 2.65 | 99.1 | Very high | Yes |
| 12 | 5 | 30.46 | 134 | Very high | Yes | 52 | 29 | 2.07 | 46 | Moderate | Yes |
| 13 | 3 | 31.93 | 87.8 | High | Yes | 53 | 40 | 2.925 | 87.4 | High | Yes |
| 14 | 3 | 24.83 | 68.3 | High | Yes | 54 | 10 | 7.44 | 62 | High | Yes |
| 15 | 10 | 27.75 | 231 | Very high | Yes | 55 | 3 | 34.43 | 94.7 | Very high | Yes |
| 16 | 3 | 50.07 | 138 | Very high | Yes | 56 | 27 | 4.52 | 94.1 | Very high | Yes |
| 17 | 9 | 32.6 | 247 | Very high | Yes | 57 | 9 | 9.22 | 69.8 | High | Yes |
| 18 | 12 | 7.92 | 78.1 | High | Yes | 58 | 3 | 25.07 | 69 | High | Yes |
| 19 | 5 | 15.3 | 67.4 | High | Yes | 59 | 32 | 5.06 | 123 | Very high | Yes |
| 20 | 7 | 28.36 | 170 | Very high | Yes | 60 | 28 | 3.36 | 72.3 | High | Yes |
| 21 | 5 | 34.92 | 154 | Very high | Yes | 61 | 27 | 9.01 | 188 | Very high | Yes |
| 22 | 3 | 39.3 | 108 | Very high | Yes | 62 | 8 | 19.99 | 136 | Very high | Yes |
| 23 | 8 | 7.04 | 47.8 | Moderate | Yes | 63 | 18 | 11.98 | 172 | Very high | Yes |
| 24 | 9 | 5.14 | 38.9 | Moderate | Yes | 64 | 5 | 20.92 | 92.1 | Very high | Yes |
| 25 | 17 | 5.45 | 74.1 | High | Yes | 65 | 43 | 5.28 | 169 | Very high | Yes |
| 26 | 9 | 8 | 60.5 | Moderate | Yes | 66 | 41 | 5.54 | 169 | Very high | Yes |
| 27 | 5 | 15.46 | 68.1 | High | Yes | 67 | 38 | 5.81 | 166 | Very high | Yes |
| 28 | 52 | 2.04 | 77.6 | High | Yes | 68 | 36 | 2.59 | 70.3 | High | Yes |
| 29 | 64 | 2.83 | 130 | Very high | Yes | 69 | 21 | 6.2 | 102 | Very high | Yes |
| 30 | 51 | 1.96 | 73.3 | High | Yes | 70 | 32 | 3.37 | 82 | High | Yes |
| 31 | 11 | 6.59 | 60 | Moderate | Yes | 71 | 6 | 15.67 | 81.6 | High | Yes |
| 32 | 14 | 4.99 | 56.7 | Moderate | Yes | 72 | 25 | 1.95 | 37.8 | Moderate | Yes |
| 33 | 54 | 3.93 | 155 | Very high | Yes | 73 | 29 | 1.7 | 37.8 | Moderate | Yes |
| 34 | 40 | 3.22 | 96.2 | Very high | Yes | 74 | 28 | 1.7 | 36.6 | Moderate | Yes |
| 35 | 34 | 2.77 | 71.3 | High | Yes | 75 | 8 | 5.74 | 39 | Moderate | Yes |
| 36 | 4 | 23.05 | 82.6 | High | Yes | 76 | 28 | 2.59 | 55.7 | Moderate | Yes |
| 37 | 5 | 17.02 | 74.9 | High | Yes | 77 | 28 | 2.05 | 44.1 | Moderate | Yes |
| 38 | 20 | 7.32 | 116 | Very high | Yes | 78 | 29 | 1.7 | 37.8 | Moderate | Yes |
| 39 | 6 | 21.92 | 114 | Very high | Yes | 79 | 5 | 17.88 | 78.7 | High | Yes |
| 40 | 7 | 8.76 | 52.6 | Moderate | Yes | 80 | 16 | 9.61 | 124 | Very high | Yes |

The comparison results of landslide determination are shown in Table 2.

TABLE 2

| | Cr < 32.6 | | 32.6 ≤ Cr < 61.7 | | 61.7 ≤ Cr < 90.8 | | Cr ≥ 90.8 | | |
|---|---|---|---|---|---|---|---|---|---|
| | Number | % | Number | % | Number | % | Number | % | Total |
| Landslides | 0 | 0 | 16 | 20.0 | 25 | 31.3 | 39 | 48.7 | 80 |

From Table 2, it can be seen that all landslide sites were characterized by moderate or higher possibilities, with Cr≥32.6; nearly half, 48.7%, of the landslide sites were characterized by a very high possibility, with Cr≥90.8; and most, 80.0%, of the landslide sites were characterized by a high possibility, with Cr≥61.7. These data indicate that the early warning method of the present disclosure can greatly reduce underreporting rate and guarantee the reliability of early warning of landslides.

1. An early warning method for a shallow soil landslide based on rainfall and soil moisture content, comprising the following steps:
a. consulting meteorological data for a location of a landslide mass to obtain a local annual average rainfall amount, local annual average sunshine duration, and a local average temperature in a rainy season from May to September, and determining whether the location is a humid region according to Equation 1;
$$Rm > 1.38 Ss - 1068 \qquad Equation 1$$
wherein Rm is the annual average rainfall amount in mm; and Ss is the annual average sunshine duration in h;
b. calculating soil moisture content of the humid region in the rainy season;
$$S = 0.0168 ln ( M ) + 0.3452 \qquad Equation 2$$
wherein S is the soil moisture content; and M is a soil moisture index, calculated by Equation 3;
$$M = R / pe \qquad Equation 3$$
wherein R is a local 24-hour rainfall amount in mm/d; and Pe is maximum latent heat evaporation in mm/d, calculated by Equation 4;
$$pe = \{ \begin{matrix}0 & {T}_{a} < 0 ° C . \\ [ 0.533 {( \frac{10 {T}_{a}}{I} )}^{a} ] \frac{h}{12} & 0 ° C . \\ - 13.86 + 1.075 {T}_{a} - 0.0144 {T}_{a}^{2} & {T}_{a} > 26.5 ° C .\end{matrix} \qquad Equation 4$$
wherein Tₐ is a monthly average temperature in ° C.; I is an annual total heating index, calculated by Equation 5; H is daylight length in h; and a is a coefficient, calculated by Equation 6;
$$I = {\sum }_{1}^{12} i \qquad Equation 5$$
$$a = 0. 492 + 1.792 \times 1 {0}^{- 2} I - 7.71 \times 1 {0}^{- 5} {I}^{2} + 6.75 \times 1 {0}^{- 7} {I}^{3} \qquad Equation 6$$
wherein i is an annual heating coefficient, calculated by Equation 7;
$$i = {( \frac{{T}_{a}}{5} )}^{1.514} \qquad Equation 7$$
c. monitoring rainfall amounts in real time by a plurality of sensors at a monitored landslide site, and obtaining a first 24-hour rainfall amount on an hourly basis;
d. calculating the soil moisture content S by step b, wherein if S<0.37, rainfall has no impact on the landslide and is disregarded, rainfall duration D=0, and the rainfall amount is 0; if S≥0.37, an onset of the rainfall is confirmed, the rainfall duration D=1 h, and a previous 1-hour rainfall amount is included in a total rainfall amount as an initial 1-hour rainfall amount; and if the soil moisture content S<0.37 again, the previous rainfall amount no longer affects the landslide, the rainfall duration D=0, and the rainfall amount is 0;
e. when D=1 h in step d, calculating the rainfall duration on an hourly basis, and calculating a total hourly rainfall amount by superposition from the initial 1-hour rainfall amount, and dividing the total hourly rainfall amount by the rainfall duration to obtain hourly rainfall intensity;
f. calculating a critical value Cr of a shallow soil landslide by Equation 8 based on the rainfall duration and the rainfall intensity;
$$C r = {I}_{i} {D}^{0.921} \qquad Equation 8$$
wherein Iᵢ is the rainfall intensity in mm/h; and D is the rainfall duration in h; and
g. performing conditional discrimination based on the critical value Cr of the shallow soil landslide obtained in step f to determine a landslide grade, and giving an early warning signal.

2. The early warning method for the shallow soil landslide based on rainfall and soil moisture content according to claim 1, wherein in step g, the landslide grade is sequentially a green early warning grade, a yellow early warning grade, an orange early warning grade, or a red early warning grade.

3. The early warning method for the shallow soil landslide based on rainfall and soil moisture content according to claim 2, wherein in step g, the early warning signal is determined by the conditional discrimination: when the critical value Cr<32.6, a possibility of a landslide is low, and a green safety signal is given; when 32.6≤Cr<61.7, the possibility is moderate, and a yellow early warning signal is given; when 61.7≤Cr<90.8, the possibility is high, and an orange early warning signal is given; and when the critical value Cr≥90.8, the possibility is very high, and a red early warning signal is given.
